(19) United States
(12) Patent Application Publication
(10) Pub. No.: US 2002/0076425 A1
(43) Pub. Date: Jun. 20, 2002

(54) Cosmetic compositions comprising at least one continuous liquid fatty phase structured with polyurethanes, polyurethaneureas, and polyureas

(76) Inventors: Jean Mondet, Aulnay Sous Bois (FR); Bertrand Lion, Luzarches (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/964,751

(22) Filed: Sep. 28, 2001

(30) Foreign Application Priority Data
Sep. 28, 2000 (FR) 00 12363

(51) Int. Cl. A61K 7/00

(52) U.S. Cl. 424/401

(57) ABSTRACT

A cosmetic composition comprising at least one continuous liquid fatty phase containing at least one hydrocarbon-based oil and structured with at least one polymer chosen from polyurethanes, poliyurethaneureas, and polyureas, said at least one polymer being soluble or dispersible in said at least one hydrocarbon-based oil, and wherein said at least one polymer comprises in the chain thereof and/or grafted thereto:

at least two groups chosen from urethane groups and urea groups, and

at least one hydrocarbon-based unit chosen from hydrocarbon blocks and grafts, and from blocks and grafts of hydrocarbon-based, long-chain aliphatic polyesters.

The present invention relates to a care and/or treatment and/or make-up composition for the skin, integuments or the lips of human beings, comprising a liquid fatty phase, structured with at least one polymer, making it possible in certain embodiments to obtain media which are gelled just up to solid form and which conserve transparency.

These compositions, which may in certain embodiments be in the form of sticks, give non-migrating, glossy or matte deposits, after application, when a suitable amount of fillers is added, which are water-resistant and rub-resistant, and have prolonged staying power with maintenance of the gloss or matte effect, as appropriate.

In general, in cosmetic or dermatological products, the liquid fatty phase is generally structured, that is to say gelled and/or rigidified, conventionally using waxes or fillers. This is the case especially for solid compositions such as deodorants, lip balms, lipsticks, concealer products, and cast foundations.

For the purposes of this patent application, the expression “liquid fatty phase” means a fatty phase which is liquid at ambient temperature (25° C.) and at atmospheric pressure (760 mmHg) composed of one or more mutually compatible fatty substances that are liquid at ambient temperature, also known as oils.

The expression “structured fatty phase” means a fatty phase which is simply thickened to solidified, or in certain embodiments gelled.

However, waxes have the major drawback of reducing the gloss of the deposit, which is not always desirable, in certain embodiments such as lipsticks.

Also, the addition of waxes to structure the liquid fatty phase also has the drawback of not always preventing the migration over time of this phase into the wrinkles and fine lines of the skin, especially around the lips and the eyes, which is particularly inconvenient for lipsticks and eyeshadows. Specifically, a large migration of the liquid fatty phase entrains dyestuffs with it, where applicable, and leads to an un-aesthetic effect around the lips, which particularly accentuates fine lines.

The term “migration” means an overflowing of the composition, and in particular of the colour, beyond the initial line of the make-up. This migration of the fatty phase is considered by lipstick users as a major defect.

Moreover, it is possible to increase the gloss by reducing the content of waxes and/or fillers in the composition. However, this results in a decrease in the hardness of the stick, and an increase in the migration of the liquid fatty phase. In other words, the contents of waxes and of fillers required to structure the liquid fatty phase, in particular to prepare a stick of suitable hardness whose fatty phase does not migrate, are a limitation on the gloss of the deposit.

U.S. Pat. No. 5,919,441 describes polysiloxanes used as gelling agents in cosmetic formulations. These polysiloxanes comprise both organosiloxane groups and polar groups which can form hydrogen interactions between themselves. These polar groups are selected from ester, urea, thiourea, and amide groups.

Although polyorganosiloxanes bearing urea groups are cited as gelling agents for silicone oils, polyorganosiloxanes have the drawback, however, of not being able to gel the hydrocarbon-based oils generally used in cosmetic compositions, in which they are neither soluble nor dispersible.

In addition, the quality of the deposits obtained using these silicone gels is unknown, in particular whether they are water-resistant and rub-resistant, whether they are capable of conserving the gloss or matte effect, depending on the case, and, finally, whether they have a tendency to migrate into the wrinkles and fine lines of the skin or the lips.

For cosmetic and/or dermatological applications, it is desirable to obtain compositions whose application leads to deposits that are resistant to water, sweat, saliva and/or tears, that do not leave marks and do not migrate, and whose staying power over time, in particular of the colour (no colour change and no fading of the colour), is as long as possible. This is especially the case for foundations, lipsticks and mascaras.

Polyurethanes and/or polyureas are known to provide film-forming properties of water-resistance and mechanical-resistance, in particular rub-resistance. In addition, they lead to the formation of films whose flexibility is readily adjustable by adapting the chemical composition. However, these polymers, which are polar due to hydrogen interactions between the chains, are not soluble in oils. It is therefore not possible to convey them in the hydrocarbon-based oil media conventionally used in cosmetics, either as a solution or as a dispersion.

The inventors' research sought a care and/or treatment and/or make-up composition for the skin and/or integuments and/or the lips which allows at least one of these drawbacks to be overcome.

The inventors have found, surprisingly, that the use of specific polymers, chosen from polyurethanes, polyurethaneureas, and polyureas, which are soluble or dispersible in hydrocarbon-based oils, gives after application deposits having at least one of the following properties:

do not migrate in the wrinkles and fine lines of the skin or the lips,

are resistant to at least one of water, sweat, saliva, sebum, and tears,

are at least one of glossy and gloss conserving over a long period of time, such as in lips and eyelash applications

are matte if a suitable amount of fillers is added thereto, and may conserve their matte effect over a long period of time, and

generally provide a prolonged staying power of the make-up.

Furthermore, these deposits may show good transfer-resistance properties, i.e., they leave few or no marks on certain supports with which they are placed in contact, such as a glass, a fabric, a cup, a cigarette, or the skin.

These polymers can structure these oils while at the same time conserving their transparency.

Certain embodiments of the invention are compositions comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:

at least two groups chosen from urethane groups and urea groups, and

at least one hydrocarbon-based unit chosen from hydrocarbon blocks and grafts, and from blocks and grafts of hydrocarbon-based, long-chain, in some embodiments branched, aliphatic polyesters.

The expression “hydrocarbon-based long-chain” includes both linear and branched hydrocarbon-based chains containing at least 8 carbon atoms and in certain embodiments ranging from 10 to 500 carbon atoms.

The polyurethanes, polyurethaneureas, and polyureas of certain embodiments of the invention have a number average molecular mass ranging from 1000 to 1,000,000, in some embodiments ranging from 2000 to 500,000 and in certain other embodiments from 3000 to 250,000, as measured at the peak height of steric exclusion chromatography (GPC).

The polymers according to certain embodiments of the invention have a number average molecular mass of greater than 10,000 but less than 250,000.

The polymers according to some embodiments of the invention are defined by one of the following three formulae:

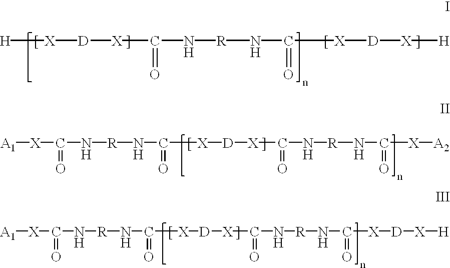

in which:

n denotes an integer ranging from 1 to 10,000 and in certain embodiments ranging from 1 to 1000,

X, which may be identical or different, is chosen from —O—, —NH—, and combinations thereof,

R, which may be identical or different, is a divalent radical chosen from alkylene, cycloalkylene, and aromatic radicals, and combinations thereof, which are optionally functionalized,

A₁ and A2, which may be identical or different, are chosen from saturated and unsaturated, linear, branched, and cyclic monovalent hydrocarbon-based radicals containing from 1 to 80 carbon atoms,

D, which may be identical or different, is chosen from

(1) saturated and unsaturated, aliphatic and cycloaliphatic hydrocarbon-based divalent blocks,

(2) hydrocarbon-based long-chain aliphatic polyester blocks,

(3) grafts

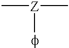

in which Z is a hydrocarbon-based trivalent radical which may contain at least one hetero atom, and φ is chosen from linear, branched, and cyclic aliphatic chains, and (4) combinations of the blocks (1) and grafts (3).

The monovalent hydrocarbon-based radicals A₁ and A₂ are, in certain embodiments, chosen from saturated and unsaturated, aliphatic, cycloaliphatic and aromatic radicals. The radicals A₁ and A₂ may, in some embodiments, be obtained from monoalcohols and/or monoamines optionally used to consume the isocyanate groups that are residual at the end of polymerization.

When D is chosen from saturated and unsaturated, aliphatic and cycloaliphatic hydrocarbon-based blocks, it may be obtained from:

natural and synthetic oils,

products of addition, such as, dimer, trimer and polymers, of at least two unsaturated aliphatic chains, such as aliphatic radicals derived from “dimeric” fatty acids, for example, the products of addition between oleic chains, and

polyenes, which are in some embodiments hydrogenated, such as polybutadiene, hydrogenated polyisoprene, polyolefins, and copolyolefins.

When D is chosen from hydrocarbon-based long-chain aliphatic polyester blocks, it is obtained in some embodiments from hydrocarbon-based long-chain branched polyesters such as, for example, poly(12-hydroxystearate).

When D is chosen from grafts, φ is chosen from linear, branched and cyclic aliphatic chains containing from 8 to 40 carbon atoms. The optional hetero atoms in the trivalent radical Z are in some embodiments chosen from —O—, —N— and —S—.

The polyurethanes, polyurethaneureas, and polyureas according to certain embodiments of the invention represent, as solids content, from 0.1% to 80% of the total weight of the composition and in some additional embodiments from 0.5% to 40% of the total weight of the composition.

Structuring polyurethanes, polyurethaneureas and polyureas according to certain embodiments of the invention can result from the polymerization reaction between:

(1) at least one diisocyanate chosen from aliphatic, cycloaliphatic and aromatic diisocyanates of general formula O═C═N—R—N═C═O, in which R, which may be identical or different, is a divalent radical chosen from alkylene, cycloalkylene and aromatic radicals, and combinations thereof, which are optionally functionalized,

(2) at least one difunctional derivative H—X—D—X—H, having two active hydrogens which can each react with an isocyanate group, in which

X, which may be identical or different, is chosen from —O—, —NH—, and combinations thereof, and

D is as defined above, and

(3) optionally, a monofunctional derivative A₁—X—H, or two monofunctional derivatives A₁—X—H and A₂—X—H, having only one active hydrogen which can react with an isocyanate group, to consume the residual isocyanate groups that have not fully reacted with the difunctional reagents H—X—D—X—H, the monofunctional derivatives A₁—X—H and A₂—X—H possibly being identical or different, and A₁ and A₂ being as defined above.

The isocyanates used in the polymerization reaction may be aliphatic, cycloaliphatic, or aromatic. Hexamethylene diisocyanate, isophorone diisocyanate, toluene diisocyanate, 4,4′-dicyclohexylmethane diisocyanate, and diisocyanates obtained from fatty acid dimers can, for example, be used.

To increase the final solubility in the hydrocarbon-based oil of the structuring polyurethanes, polyurethaneureas and polyureas according to some embodiments of the invention, it is also possible to use the diisocyanates obtained from dimeric fatty acids. These diisocyanates from dimeric fatty acids are described in the article R. Höfer, Oleochemical polyols. New Raw Materials for Polyurethane Applications, Eur. Coating J., Mar. 2000, at 26-37.

The difunctional derivatives H—X—D—X—H may be chosen from diol dimers and derivatives thereof, alkane diols, polyolefins with hydroxyl ends, such as polydienes with hydroxyl ends, which are, in some embodiments of the invention, hydrogenated, long-alkyl-chain branched polyesters bearing at least two reactive groups, natural and synthetic oils bearing two to three hydroxyl groups, and finally long-aliphatic-chain diamines and diamine dimers.

The diol dimers of certain embodiments are chosen from branched C₃₆ aliphatic and alicyclic diols. These diols may be prepared from the “corresponding dimeric fatty acids”.

The expression “corresponding dimeric fatty acids” means dimeric fatty acids which have the same structure as these diols, but which have two carboxylic acid ends instead of diol ends.

As mentioned in Höfer, the conversion of the dimeric fatty acids into diol dimers may be carried out either by hydrogenation of methyl esters of the dimeric fatty acids or by direct dimerization of the oleic alcohol.

In certain embodiments of the invention, use may be made of the diol dimers sold by the company Cognis under the trade names Sovermol 908 (at 97% purity) and Sovermol 650 NS (at 68% purity).

It is also possible, in some embodiments, to use polyether diol oligomers and polycarbonate diol oligomers, prepared by subsequent etherification or esterification of these same branched C₃₆ diol dimers. These oligomers generally have a number-average molecular mass ranging from 500 to 2000, and contain two hydroxyl functions.

The polydienes with hydroxyl ends of certain embodiments may be, for example, those defined in French patent FR-2 782 723, the disclosure of which is specifically incorporated by reference herein. In some embodiments, the polydienes are chosen from the group of polymers formed from at least one monomer chosen from butadiene, isoprene and 1,3-pentadiene. These polydienes have a number-average molecular mass of less than 7000 and in some embodiments ranging from 1000 to 5000. The polydienes of certain embodiments have a chain-end functionality ranging from 1.8 to 3 and in certain additional embodiments in the region of 2. These polydienes with hydroxyl ends are, for example, the hydroxylated polybutadienes sold by the company Elf Atochem under the brand names Poly BD-45H® and Poly BD R-20 LM®. These products are hydrogenated as used in some embodiments.

In some embodiments, it is possible to use polyolefin homopolymers or copolymers with α,ω-hydroxyl ends, chosen from:

polyisobutylene oligomers with α,ω-hydroxyl ends, and

the copolymers sold by the company Mitsubishi under the brand names Polytail®, in particular those of structure:

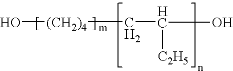

with a melting point of 60 to 70° C.

It is possible in certain embodiments to use as difunctional derivative H—X—D—X—H, a long-alkyl-chain branched polyester comprising at least two reactive groups, for example, poly(12-hydroxystearate) containing hydroxyl ends. This polyester is obtained by self-condensation of 12-hydroxystearic acid, followed by reaction with a polyol to consume the residual acid groups.

Certain embodiments of the invention make use of oligomer of structure

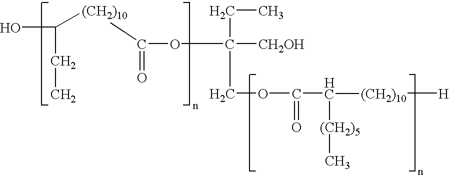

in which the sum m+n is such that the oligomer has a number-average molecular mass in the region of 2000 and a hydroxyl functionality in the region of 1.8.

Natural and synthetic oils bearing two to three hydroxyl groups may also be used as difunctional derivative H—X—D—X—H in certain embodiments.

In some embodiments of the invention, the at least one oil used will be chosen from those bearing two hydroxyl groups per chain, and in certain additional embodiments, chosen from monoglycerides of structures (1) and (2):

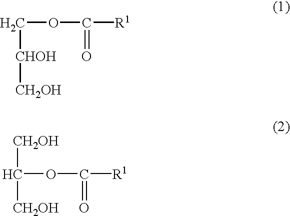

R¹ being chosen from linear and branched C₈ to C₃₀ alkyl chains such as, for example, glyceryl monostearates.

Such glyceryl monostearates correspond, for example, to the difunctional derivatives H—X—D—X—H, in which:

D represents

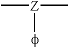

X represents —O—, and

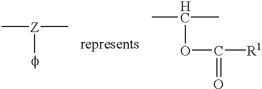

in which R¹ is defined as above.

When these glycerol monoesters of some embodiments of the invention are reacted with a diisocyanate, a solubilizing graft is introduced into the polymer chain rather than a block, as in the difunctional derivatives mentioned above.

In certain embodiments of the invention, the difunctional derivative H—X—D—X—H chosen from oils bearing three hydroxyl groups per chain, for example, hydrogenated and non-hydrogenated castor oils, will be used.

In certain embodiments of the invention, the polymerization reaction is carried out with a deficit of diisocyanate relative to the reaction stoichiometry, to avoid the crosslinking of the polymer and to conserve good solubility thereof.

Long-chain aliphatic diols may also be used in certain embodiments of the invention. In some embodiments, diols of structure HO—D—OH in which D is chosen from linear and branched alkyl chains containing from 8 to 40 carbon atoms will be used. These diols are sold by the company Atochem under the name Vikinol®. Some embodiments make use of 1,12-dodecanediol and 1,10-decanediol, the latter being sold by the company Cognis under the trade name Sovermol 110®.

It is possible in certain embodiments of the invention to use diols of structure

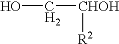

in which R² is chosen from alkyl chains containing from 8 to 40, such as 8 to 30, carbon atoms.

In certain embodiments of the invention, these long-chain aliphatic diols are used with any one of the H—X—D—X—H derivatives mentioned above, to serve as chain couplers during the synthesis of polyurethanes, polyurethaneureas and polyureas.

In some additional embodiments, long-chain aliphatic diamines and diamine dimers may be used as difunctional derivatives H—X—D—X—H.

The use of such reagents in the polymerization reaction makes it possible to introduce into the polymer urea groups rather than urethane groups.

According to certain embodiments of the invention, diamine dimers having the same structure as the diol dimers mentioned above will be used, i.e. diamine dimers comprising two primary amine functions instead of hydroxyl groups.

These diamine dimers of some embodiments may be obtained from the conversion of dimeric fatty acids, for instance the diol dimers in Höfer.

In some additional embodiments, diamines of structure H₂N—D—NH₂ in which D is chosen from linear and branched alkyl chains containing from 8 to 40 carbon atoms may be used. These diamines are used in some embodiments as a mixture with a difunctional derivative H—X—D—X—H chosen from diol dimers and derivatives thereof, polyolefins, such as polydienes, with hydroxyl ends, long-alkyl-chain branched polyesters, and oils bearing two to three hydroxyl groups.

Certain embodiments of the invention make use of diamines chosen from:

1,10-diaminodecane and 1,12-diaminododecane, and

the following diamino oils sold by the company Akzo Nobel: cocopropylene diamines (distilled and undistilled) Duomeen® C and CD, hydrogenated tallowpropylene diamine Duomeen® HT, C₁₆₋₂₂ alkylpropylene diamine Duomeen® M, oleylpropylene diamine Duomeen® 0, tallowpropylene diamine Duomeen®) T.

The monofunctional derivatives A₁—X—H and A₂—X—H in certain embodiments are chosen from linear and branched monoalcohols and monoamines having alkyl chains containing from 1 to 80 carbon atoms, natural and synthetic oils bearing a single hydroxyl group per chain, for example, glycerol diesters, and citric acid triesters of fatty alcohols.

According to certain embodiments of the invention, the polymerization reaction may also be carried out in the presence of additional reagents chosen from

(a) polyorganosiloxanes of structure:

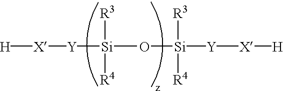

in which

Z denotes an integer ranging from 0 to 100,

X′, which may be identical or different, is chosen from —O—- and —NH—,

Y, which may be identical or different, is chosen from hydrocarbon-based radicals of structure —(CH2)ₐ—, with “a” ranging from 0 to 10, and

R³ and R⁴, which may be identical or different, are groups chosen from methyl, phenyl, alkyl, perfluoroalkyl, and polyoxyalkylene groups,

(b) polyethers of structure H—X′—E—X′—H, in which

X′, which may be identical or different, is chosen from —O—and —NH—, and

E is chosen from polyoxyalkylene chains,

(c) polyesters of structure HO—F—OH in which F is a block chosen from aliphatic and aromatic polyester blocks,

(d) polyamides of structure H₂N—G—NH₂ in which G is chosen from polyamide blocks, such as in certain embodiments, polyamides resulting from the reaction of a fatty acid dimer and of a diamine, which are in some embodiments aliphatic,

(e) polyorganosiloxanes comprising a single difunctional end, of structure

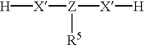

in which

X′, which may be identical or different, is chosen from —O—and —NH—,

Z is chosen from hydrocarbon-based trivalent radicals which may contain at least one hetero atom, such as oxygen, sulfur, and nitrogen, and

R⁵ is chosen from polyorganosiloxane segments, (f) difunctional coupling agents of structure H—X′—J—X′—H in which

X′, which may be identical or different, is chosen from —O—and —NH—, and

J is chosen from linear, branched and cyclic hydrocarbon-based divalent radicals which may bear hydrophilic groups, such as carboxylic acid, sulfonic acid, tertiary amine and polyoxyalkylene groups, in certain embodiments.

The polycondensation reactions envisaged by certain embodiments of the invention are conventionally carried out in an organic solvent capable of dissolving the reagents and the polymer formed. In other embodiments, this solvent is readily removable at the end of the reaction, in certain embodiments by distillation, and does not react with the isocyanate groups.

In some embodiments, each of the reagents is dissolved in some of the organic solvent before the polymerization reaction.

It is desired in certain embodiments to use a catalyst to activate the polymerization. The catalyst of certain embodiments will be chosen from the catalysts commonly used in polyurethane and polyurea chemistry, such as, for example, tin 2-ethylhexanoate.

The molar proportion between the main reagents of the polymerization reaction depends on the chemical structure and on the molecular weight of the polymers (polyurethanes, polyurethaneureas, and polyureas) which it is desired to obtain, as is conventionally the case in polyurethane and polyurea chemistry. Similarly, the order of introduction of the reagents will be adapted to this chemistry.

Thus, in some embodiments, the reaction of two moles of functional derivative H—X—D—X—H with one mole of diisocyanate gives, after total consumption of the reagents, a polymer defined by formula I:

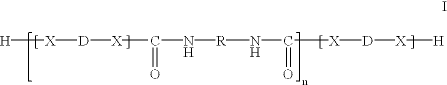

wherein X, D, R, and n are as defined above.

For this reaction, the process of certain embodiments will be performed in the following manner:

the initial medium is a solution comprising two moles of derivative H—X—D—X—H, for example two moles of diol dimer, in a solvent, for example tetrahydrofuran,

a solution comprising one mole of diisocyanate dissolved in the same solvent, for example, toluene diisocyanate dissolved in tetrahydrofuran, is added dropwise to this initial solution.

Moreover, in some embodiments, the equimolar reaction of a difunctional derivative H—X—D—X—H with a diisocyanate, with consumption of the residual isocyanates by a monofunctional compound A₁—X—H, gives a polymer defined by formula III:

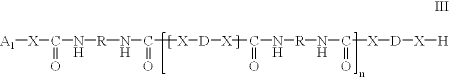

wherein X, R, D, and n are as defined above.

In some embodiments, this reaction will then preferably be carried out by simultaneous addition, into a reactor, of an organic solution of one mole of H—X—D—X—H, for example, a Polytail® described above, and of an organic solution of one mole of diisocyanate, for example, 4,4′-dicyclohexylmethane diisocyanate. The simultaneous addition of these two organic solutions is also known as “double addition”. Because both reagents are added in the same molar quantity, it is an “equimolar” reaction.” At the end of the double addition, the reaction mixture is heated at 60° C. for 5 hours. A sample of the reaction medium is then taken to assay the residual isocyanates using a method known to those skilled in the art. Finally, a solution of a chosen monofunctional compound A₁—X—H is added to the reaction medium, in an amount which is sufficient to consume the residual isocyanates, this amount having been estimated from the assay of the residual isocyanates. In certain embodiments, 1-Decanol will be used as monofunctional derivative A₁—X—H.

Finally, in certain other embodiments, at least one polymer is the product of the reaction between

one mole of compound H—X—D—X—H, for example, a diol dimer,

three moles of diisocyanate, for example, 4,4′-dicyclohexylmethane diisocyanate, and

two moles of coupler of structure

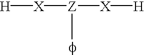

in which φ is chosen from linear, branched, and cyclic aliphatic chains containing from 8 to 20 carbon atoms, leads to the formation of the grafted, block polymer of formula:

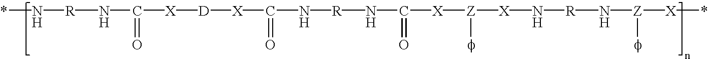

wherein R, X, D, Z, φ, and n are as defined above.

Any residual isocyanates may be consumed by adding a suitable amount of monofunctional reagent A₁—X—H.

To obtain such a polymer, the process of some embodiments of the invention is performed in the following manner:

the initial reaction medium comprises a solution comprising one mole of a difunctional derivative H—X—D—X—H,

a solution of three moles of diisocyanate is added dropwise to this medium,

the mixture is then left to react for 3 hours at 60° C.,

next, an organic solution comprising two moles of a coupler defined by the formula:

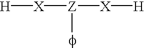

is added to this medium, any residual isocyanates possibly being consumed by adding a suitable amount of monofunctional reagent A₁—X—H.

The fatty phase of certain embodiments of the invention contains at least one hydrocarbon-based oil and optionally at least one oil, which may or may not be hydrocarbon-based.

The oils of the fatty phase of the compositions of some embodiments of the invention may be chosen from polar and non-polar, volatile and non-volatile oils which may be used in cosmetics.

In certain embodiments of the invention, the at least one hydrocarbon-based oil is chosen from polar oils comprising at least one functional group chosen from esters, ethers, acids and alcohols such as, for example:

hydrocarbon-based plant oils with a high triglyceride content consisting of fatty acid esters of glycerol in which the fatty acids may contain varied chain lengths, these chains possibly being chosen from linear and branched, and saturated and unsaturated; these oils are, in some embodiments, chosen from wheatgerm oil, corn oil, sunflower oil, carite oil, castor oil, sweet almond oil, macadamia oil, apricot oil, soybean oil, rapeseed oil, cotton oil, alfalfa oil, poppy oil, pumpkin oil, sesame oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passionflower oil and musk rose oil; or alternatively caprylic/capric acid triglycerides such as those sold by the company Stearineries Dubois or those sold under the names Miglyol 810, 812 and 818 by the company Dynamit Nobel;

synthetic oils of formula R⁶COOR⁷ in which R⁶ is chosen from linear and branched higher fatty acid residues containing from 7 to 19 carbon atoms and R⁷ is chosen from branched hydrocarbon-based chains containing from 3 to 20 carbon atoms, for example, Purcellin oil (cetostearyl octanoate), isononyl isononanoate C12 to C15 alkyl benzoates;

synthetic esters and ethers, for instance isopropyl myristate, 2-ethylhexyl palmitate, alkyl and polyalkyl octanoates, decanoates and ricinoleates, hydroxylated esters, for instance isostearyl lactate and diisostearyl malate, and pentaerythritol esters;

C₈ to C₂₆ fatty alcohols, for instance oleyl alcohol; and

combinations thereof.

In certain additional embodiments the at least one hydrocarbon-based oil is chosen from apolar oils chosen from volatile and non-volatile, linear and cyclic silicone oils that are liquid at ambient temperature, such as:

polydimethylsiloxanes (PDMSs), comprising groups chosen from alkyl, alkoxy, and phenyl groups that are pendent and/or at the end of the silicone chain and containing from 2 to 24 carbon atoms;

phenylsilicones, for instance phenyl trimethicones, phenyl dimethicones, phenyl trimethylsiloxydiphenylsiloxanes, diphenyl dimethicones, diphenyl methyidiphenyl trisiloxanes and 2-phenylethyl trimethylsiloxysilicates;

linear and branched hydrocarbons and fluorocarbons of synthetic and mineral origin, for instance volatile oils, such as liquid paraffins (for example isoparaffins) and aliphatic hydrocarbons (for example isododecane), and non-volatile oils and derivatives thereof, petroleum jelly, polydecenes, hydrogenated polyisobutene, such as parleam, and squalane, and combinations thereof.

In some embodiments of the invention, the structured oils, and in certain additional embodiments, oils structured with polyurethanes, polyurethaneureas, and polyureas, are apolar oils of the hydrocarbon-based type of mineral and synthetic origin, chosen in certain embodiments from hydrocarbons, in certain embodiments chosen from alkanes, for instance parleam oil, isoparaffins including isododecane, and squalane, and mixtures thereof.

Although the polymers according to certain embodiments of the invention cannot gel all silicone oils alone, whether they are volatile or non-volatile, they can gel mixtures of silicone oil and of hydrocarbon-based oils, the hydrocarbon-based oils being chosen, in some embodiments, from the oils mentioned above. These mixtures of oils comprise from 0% to about 40% by weight of silicone oils in some embodiments.

Although the structuring polymers of the invention can gel the oils of the fatty phases of the compositions of certain embodiments of the invention up to the solid state without using wax, it is possible in some embodiments, to add waxes to the compositions.

For the purposes of some embodiments of the present invention, the wax is a lipophilic fatty compound that is solid at ambient temperature (25° C.), with a melting point of greater than 40° C. which may be up to 200° C.

The waxes which may be used in certain embodiments are those used in the cosmetic and dermatological fields. They are, in certain embodiments, of natural origin, for instance beeswax, carnauba wax, candelilla wax, ouricoury wax, Japan wax, cork fibre wax, or sugar cane wax, paraffin wax, lignite wax, microcrystalline waxes, lanolin wax, montan wax, ozokerites and hydrogenated oils, for instance hydrogenated jojoba oil, and of synthetic origin, for instance the polyethylene waxes derived from the polymerization of ethylene, the waxes obtained by Fischer-Tropsch synthesis, fatty acid esters and glycerides that are solid at 40° C., and silicone waxes, for instance alkyl, alkoxy, and/or esters of poly(di)methylsiloxane that are solid at 40° C.

Nevertheless, the compositions according to some embodiments of the invention are wax-free.

In some embodiments, the change of solid-liquid state of a wax is reversible, and this wax has in the solid form an anisotropic crystal organization, which gives the composition a cloudy, more or less opaque appearance. By bringing the wax to its melting point, it is possible in certain embodiments to make it miscible with oils and to form a microscopically homogeneous mixture. However, when this mixture is then cooled to ambient temperature, a recrystallization of the wax in the oils of the mixture takes place, resulting in a decrease in the gloss of the composition.

The term “wax-free composition” means a composition comprising less than 0.5% by weight of wax relative to the total weight of the composition.

The composition of certain embodiments of the invention may also comprise any additive usually used in the field under consideration, chosen from water optionally thickened or gelled with an aqueous phase thickener or gelling agent, antioxidants, essential oils, preserving agents, fragrances, neutralizers, liposoluble polymers, fillers, cosmetic or dermatological active agents, for example, emollients, moisturizers, vitamins, essential fatty acids, and sunscreens, and mixtures thereof. These additives may be present in the composition of some embodiments in a proportion ranging from 0 to 20% of the total weight of the composition, and in certain other embodiments, ranging from 0 to 10%. The composition is filler-free in some embodiments.

In some embodiments of the invention, the composition contains at least one agent chosen from cosmetic and dermatological active agents.

Needless to say, a person skilled in the art will take care to select the optional additional additives and the amount thereof, such that the properties of the composition according to certain embodiments of the invention are not, or are not substantially, adversely affected by the addition envisaged.

The composition according to certain embodiments of the invention may also contain a dyestuff. The dyestuff of some embodiments may be chosen from the lipophilic dyes, hydrophilic dyes, pigments, and nacres usually used in cosmetic or dermatological compositions, and mixtures thereof. The dyestuff of certain embodiments is present in a proportion ranging from 0.01% to 40% relative to the total weight of the composition, and in some embodiments ranging from 5% to 25%.

The liposoluble dyes are, for example, Sudan red, DC Red 17, DC Green 6, β-carotene, soybean oil, Sudan brown, DC Yellow 11, DC Violet 2, DC orange 5, or quinoline yellow. These liposoluble dyes of certain embodiments of the invention are present in amounts ranging from 0 to 20% of the weight of the composition and in still other embodiments, are present in amounts ranging from 0.1% to 6% of the weight of the composition.

The pigments used in certain embodiments may be chosen from white and coloured, mineral, and organic, and coated and uncoated pigments. In certain embodiments of the invention, the pigment used is a mineral pigment chosen from titanium dioxide, which may be optionally surface-treated, zirconium oxide or cerium oxide, iron oxide, chromium oxide, manganese violet, ultramarine blue, chromium hydrate, and ferric blue. In some embodiments of the invention, the pigment used is an organic pigment chosen from carbon black, pigments of D & C type, and lacquers based on cochineal carmine or on barium, strontium, calcium or aluminium. The pigments of some embodiments of the invention may be present in amounts ranging from 0 to 40% of the total weight of the composition and in some additional embodiments may be present in amounts ranging from 2% to 25%.

The nacreous pigments in some embodiments of the invention may be chosen from white nacreous pigments, such as mica coated with titanium or with bismuth oxychloride, coloured nacreous pigments such as titanium mica with iron oxides, titanium mica with, in certain embodiments, ferric blue or chromium oxide, titanium mica with an organic pigment of the abovementioned type, and also nacreous pigments based on bismuth oxychloride. The nacreous pigments of some embodiments may be present in amounts ranging from 0 to 20% of the total weight of the composition, and the pigments of certain embodiments may be present in amounts ranging from 0.1% to 15%.

When the composition of certain embodiments of the invention is anhydrous, it may be a thickened to solidified solution, and the composition of some embodiments may be a gelled solution.

On the other hand, when the composition of some embodiments of the invention contains water, it is in the form of an emulsion, in some embodiments in the form of a water-in-oil (W/O) emulsion, whose consistency is that of thickened to solidified emulsions, and in certain embodiments, that of gelled emulsions. W/O emulsions have the advantage of conserving the gelation of the emulsion.

The composition of certain embodiments the invention may be packaged in the form of a stick or a dish.

When the fatty phase of some embodiments is gelled, it has a rigid structure, which may take the form of a tube or stick. These sticks lead, after application, to deposits having at least one of the following properties:

glossy and capable of conserving their gloss over a long period of time, matte when a suitable amount of fillers capable of conserving their matte effect over a long period of time are added to the composition, in certain embodiments for foundation uses,

do not migrate into the wrinkles and fine lines of the skin, in some embodiments wherein the composition of the embodiment is used in the areas surrounding the lips and the eyes, this being of advantage for uses for the lips and the eyelashes,

capable of resisting at least one of water, sweat, sebum, saliva, and tears,

prolonged staying power of the make-up, where appropriate, and

do not transfer and/or leave marks on a support placed in contact with the deposits.

The composition according to certain embodiments of the invention may be manufactured by the known processes generally used in cosmetics and dermatology.

The composition according to some embodiments of the invention may be in the form of:

a dermatological or care composition for keratin materials such as the skin (care creams for the skin) and/or the lips (lip balms, for protecting the lips against the cold and/or the sun and/or the wind) and/or integuments (care creams for the nails or the hair), or

a body hygiene composition such as a deodorant or make-up-removing product, packaged as a stick.

The composition of certain embodiments of the invention may be in the form of a coloured make-up product optionally having care or treatment properties, which may be applied to the skin (foundation or semi-permanent tattoo), the eyes (eyeliner), the lips (lipstick), integuments, in some embodiments for the eyelashes, the eyebrows, and the hair (cream mascara (emulsion) or anhydrous mascara).

Needless to say, the composition of some embodiments of the invention must be cosmetically or dermatologically acceptable, i.e. it may contain a physiologically acceptable non-toxic medium and may be able to be applied to human skin, integuments, or lips.

Certain embodiments of the invention are cosmetic care and/or treatment and/or make-up processes for the keratin materials of human beings, and in some embodiments for the skin, the lips, and integuments, comprising the application to the keratin materials of the composition, in certain embodiments the cosmetic composition, as defined above.

Additional embodiments of the invention comprise the use of at least one polymer chosen from polyurethanes, polyurethaneureas, and polyureas, that are soluble or dispersible in at least one hydrocarbon-based oil, and wherein the at least one polymer comprises in the chain thereof and/or grafted thereto:

at least two groups chosen from urethane groups and urea groups, and

at least one hydrocarbon-based unit chosen from hydrocarbon blocks and grafts, and from blocks and grafts of hydrocarbon-based, long-chain aliphatic polyesters,

in a cosmetic composition comprising a continuous liquid fatty phase, to structure the said fatty phase.

Still other embodiments of the invention are the use of a continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein the at least one polymer comprises in the chain thereof and/or grafted thereto:

at least two groups chosen from urethane groups and urea groups, and

at least one hydrocarbon-based unit chosen from hydrocarbon blocks and grafts, and from blocks and grafts of hydrocarbon-based, long-chain aliphatic polyesters,

for the manufacture of a physiologically acceptable composition which gives, after application, deposits that are at least one of migration-resistant, resistant to at least one of water, sweat, sebum, saliva, and tears, transfer-resistant, and prolonged staying power.

Certain embodiments of the invention are illustrated in greater detail in the examples which follow. As used herein, the phrase “at least one” means one or more.

The amounts are given on a weight basis.

### EXAMPLE 1

Preparation of a block polyurethane

| | |
|---|---|
| Sovermol ® 908 diol dimer from the company Cognis | 168 g |
| Tetrahydrofuran (THF) | 250 g |
| Mixture of: | |
| methylenebis(4-cyclohexyl isocyanate) Desmodur ® W | 39.5 g |
| from the company Bayer | |
| tetrahydrofuran (THF) | 100 g |
| Tin 2-ethylhexanoate | 0.45 g |

Mixture of:

methylenebis(4-cyclohexyl isocyanate) Desmodur® W

| | | |
|---|---|---|
| from the company Bayer | 39.5 | g |
| tetrahydrofuran (THF) | 100 | g |
| Tin 2-ethylhexanoate | 0.45 | g |

The Sovermol® 908 and the tetrahydrofuran (THF) are introduced into a 1-liter reactor equipped with a central stirrer, a nitrogen inlet, a thermometer, a condenser, and an addition funnel. The mixture is stirred and brought to the reflux temperature of the THF at a temperature of 67° C.

The mixture of Desmodur® W and THF is then introduced via the funnel, over 30 minutes.

The mixture is left to react for 3 hours at reflux, and the tin 2-ethylhexanoate is then added.

This reaction mixture is left for a further 12 hours in the refluxing THF and is then cooled to ambient temperature and precipitated in 5 liters of water.

A polymer of structure defined by formula I is finally obtained. This polymer is in the form of a white paste, which is dried under vacuum to constant weight.

### EXAMPLE 2

Preparation of a grafted block polymer

| | |
|---|---|
| methylenebis(4-cyclohexyl isocyanate) Desmodur ® W | 39.3 g |
| from the company Bayer | |
| Tetrahydrofuran (THF) | 100 g |
| Mixture of: | |
| Sovermol ® 908 diol dimer from the company Cognis | 28 g |
| Tetrahydrofuran (THF) | 100 g |
| Mixture of: | |
| Vikinol ® 24-28 from the company Atochem | 40 g |
| Tetrahydrofuran (THF) | 100 g |
| Tin 2-ethylhexanoate | 0.45 g |

The Desmodur® W and the tetrahydrofuran (THF) are introduced into the same reactor as that used in Example 1, at a temperature of 67° C.

The mixture is stirred and brought to the reflux temperature of the THF, at a temperature of 67° C.

The mixture of Sovermol® 908 and of THF is then introduced, via the funnel, over 30 minutes.

The mixture is left to react for one hour at 67° C., and the mixture of Vikinol® 24-28 and of THF is then introduced.

The mixture is left reacting for 3 hours at reflux, and the tin 2-ethylhexanoate is then added.

The mixture is left for a further 12 hours in the refluxing THF and is then cooled to ambient temperature.

The reaction mixture is then precipitated from 5 liters of water.

A grafted block polymer of formula 11 is finally obtained, which is in the form of a whitish paste which is dried under vacuum to constant weight.

### EXAMPLE 3

Manufacture of a lipstick according to the invention

Starting Materials:

| | |
|---|---|
| grafted block polymer of formula II | 25% |
| hydrogenated polyisobutene oil (Parleam sold by the | 66% |
| company Nippon Oil Fats) | |
| pigments (brown iron oxide + titanium oxide) | 9% |

Preparation:

The polymer of formula II is dissolved in the Parleam oil at 100° C., and the pigments are then added. The mixture is then mixed using a deflocculating turbomixer (Ragnerie) and then poured into lipstick moulds.

A stick of lipstick with a hardness of 400 g measured using a TA-XTZ analyser at 20° C. is obtained.

The lipstick obtained is glossy, migration-resistant, has good staying power over time and is resistant to water and saliva.

### EXAMPLE 4

Anhydrous eyeshadow according to the invention, containing inter alia:

| | |
|---|---|
| grafted block polymer of formula II | 25% |
| parleam oil | 35.1% |
| glyceryl oleate | 31.25% |

This eyeshadow in the form of a stick was prepared as in Example 3. It is glossy, has good staying power over time, is migration-resistant and resistant to water, sweat, and sebum.

What is claimed is:

1. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

2. A cosmetic composition according to claim 1, wherein at least one polymer has a number-average molecular mass ranging from 1000 to 1,000,000 as measured at the peak height of steric exclusion chromatography (GPC).

3. A cosmetic composition according to claim 2, wherein at least one polymer has a number-average molecular mass ranging from 2000 to 500,000, as measured at the peak height of steric exclusion chromatography (GPC).

4. A cosmetic composition according to claim 2, wherein at least one polymer has a number-average molecular mass ranging from 3000 to 250,000, as measured at the peak height of steric exclusion chromatography (GPC).

5. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer is chosen from polymers of formulae I, II, and III 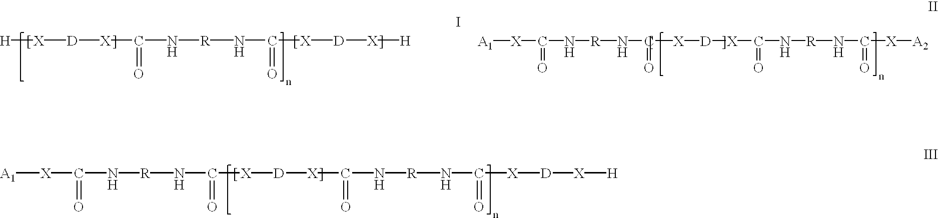
in which:
n denotes an integer ranging from 1 to 10,000,
X, which may be identical or different, is chosen from —O—, —NH—, and combinations thereof,
R, which may be identical or different, is a divalent radical chosen from alkylene, cycloalkylene and aromatic radicals, and combinations thereof, which are optionally functionalized
A₁ and A₂, which may be identical or different, are chosen from saturated and unsaturated, linear, branched, and cyclic monovalent hydrocarbon-based radicals, containing from 1 to 80 carbon atoms,
D, which may be identical or different, is chosen from
(1) saturated and unsaturated, aliphatic and cycloaliphatic hydrocarbon-based divalent blocks,
(2) hydrocarbon-based, long-chain aliphatic polyester blocks,
(3) grafts 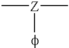
in which Z is chosen from hydrocarbon-based trivalent radicals which may contain at least one hetero atom, and φ is chosen from linear, branched, and cyclic aliphatic chains, and
(4) combinations of the blocks (1) and the grafts (3).

6. A cosmetic composition according to claim 5, wherein n denotes an integer ranging from 1 to 1000.

7. A cosmetic composition according to claim 5, wherein D is a hydrocarbon-based block obtained from:
natural and synthetic oils,
products of addition of at least two unsaturated aliphatic chains, and
polyenes.

8. A cosmetic composition according to claim 7, wherein said polyenes are hydrogenated.

9. A cosmetic composition according to claim 5, wherein D is chosen from hydrocarbon-based, long-chain aliphatic polyester blocks obtained from hydrocarbon-based long-chain branched polyesters.

10. A cosmetic composition according to claim 1, wherein said at least one polymer results from a polymerization reaction of:
(a) at least one diisocyanate chosen from aliphatic, cycloaliphatic, and aromatic diisocyanates of general formula O═C═N—R—N═C═O, in which R, which may be identical or different, is a divalent radical chosen from alkylene, cycloalkylene, and aromatic radicals, and combinations thereof, which are optionally functionalized,
(b) at least one difunctional derivative H-X-D-X-H, having two active hydrogens which can each react with an isocyanate group, in which
X, which may be identical or different, is chosen from —O—, —NH—, and combinations thereof, and
D, which may be identical or different, is chosen from
(1) saturated and unsaturated, aliphatic and cycloaliphatic hydrocarbon-based divalent blocks,
(2) hydrocarbon-based, long-chain aliphatic polyesters,
(3) grafts 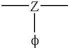
in which Z is chosen from hydrocarbon-based trivalent radicals which may contain at least one hetero atom, and φ is chosen from linear, branched, and cyclic aliphatic chains, and
(4) combinations of the blocks (1) and the grafts (3), and
(c) optionally, a monofunctional derivative A₁—X—H, or two monofunctional derivatives A₁—X—H and A₂—X—H, having only one active hydrogen which can react with an isocyanate group, to consume residual isocyanate groups that have not fully reacted with the difunctional reagents H—X—D—X—H, the monofunctional derivatives A₁—X—H and A₂—X—H possibly being identical or different, and A₁ and A₂, which may be identical or different, are chosen from saturated and unsaturated, linear, branched and cyclic monovalent hydrocarbon-based radicals, containing from 1 to 80 carbon atoms.

11. A cosmetic composition according to claim 10, wherein said diisocyanate is chosen from hexamethylene diisocyanate, isophorone diisocyanate, toluene diisocyanate, 4,4′-dicyclohexylmethane diisocyanate, and diisocyanates obtained from fatty acid dimers.

12. A cosmetic composition according to claim 10, wherein said difunctional derivative H—X—D—X—H is chosen from diol dimers and derivatives thereof, alkane diols, polyolefins with hydroxyl ends, which are optionally hydrogenated, long-alkyl-chain branched polyesters bearing at least two reactive groups, natural and synthetic oils bearing two to three hydroxyl groups, and long-aliphatic-chain diamines and diamine dimers.

13. A cosmetic composition according to claim 12, wherein said difunctional derivative H—X—D—X—H is chosen from diol dimers and derivatives thereof.

14. A cosmetic composition according to claim 12, wherein said difunctional derivative H—X—D—X—H is chosen from alkane diols.

15. A cosmetic composition according to claim 12, wherein said difunctional derivative H—X—D—X—H is chosen from polyolefins with hydroxyl ends.

16. A cosmetic composition according to claim 12, wherein said difunctional derivative H—X—D—X—H is chosen from polydienes with hydroxyl ends.

17. A cosmetic composition according to claim 12, wherein said difunctional derivative H—X—D—X—H is chosen from polydienes with hydrogenated hydroxyl ends.

18. A cosmetic composition according to claim 12, wherein said difunctional derivative H—X—D—X—H is chosen from long-alkyl-chain branched polyesters bearing at least two reactive groups.

19. A cosmetic composition according to claim 12, wherein said difunctional derivative H—X—D—X—H is chosen from natural oils bearing two to three hydroxyl groups.

20. A cosmetic composition according to claim 12, wherein said difunctional derivative H—X—D—X—H is chosen from long-aliphatic-chain diamines and diamine dimers.

21. A cosmetic composition according to claim 13, wherein said diol dimers are chosen from branched C₃₆ aliphatic and alicyclic diols, and mixtures of derivatives obtained from the conversion of corresponding dimeric fatty acids.

22. A cosmetic composition according to claim 12, wherein said difunctional derivative H—X—D−X—H is chosen from polyether diol oligomers and polycarbonate diol oligomers of number-average molecular mass ranging from 500 to 2000.

23. A cosmetic composition according to claim 16, wherein said difunctional derivative H—X—D—X—H are chosen from polydienes with hydroxyl ends formed from at least one monomer chosen from butadiene, isoprene, and 1,3-pentadiene.

24. A cosmetic composition according to claim 23, wherein said polydienes have a number-average molecular mass of less than 7000.

25. A cosmetic composition according to claim 24, wherein said polydienes have a number-average molecular mass ranging from 1000 to 5000.

26. A cosmetic composition according to claim 23, wherein said polydienes have a chain-end functionality ranging from 1.8 to 3.

27. A cosmetic composition according to claim 26, wherein said polydienes have a chain-end functionality in the region of 2.

28. A cosmetic composition according to claim 10, wherein said difunctional derivative H—X—D—X—H is chosen from polyolefin homopolymers and copolymers with α,ω-hydroxyl ends, chosen from
polyisobutylene oligomers with α,ω-hydroxyl ends, and
copolymers of structure: 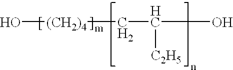
having a melting point of 60 to 70° C.

29. A cosmetic composition according to claim 12, wherein said difunctional derivative H—X—D—X—H is chosen from long-chain-alkyl branched polyesters comprising at least two reactive groups.

30. A cosmetic composition according to claim 12, wherein said difunctional derivative H—X—D—X—H is chosen from at least one natural and synthetic oil having two to three hydroxyl groups.

31. A cosmetic composition according to claim 30, wherein said at least one natural and synthetic oil is chosen from natural and synthetic oils bearing two hydroxyl groups per chain.

32. A cosmetic composition according to claim 31, wherein said at least one natural and synthetic oil is chosen from monoglycerides of structures (1) and (2) 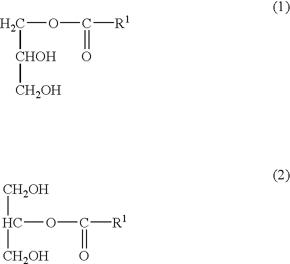
R¹ being chosen from linear and branched C₈ to C₃₀ alkyl chains.

33. A cosmetic composition according to claim 30, wherein said at least one natural and synthetic oil is chosen from natural and synthetic oils bearing three hydroxyl groups per chain.

34. A cosmetic composition according to claim 10, wherein said difunctional derivative H—X—D—X—H is chosen from diols of structure HO—D—OH in which D is chosen from linear and branched alkyl chains containing from 8 to 40 carbon atoms.

35. A cosmetic composition according to claim 10, wherein said difunctional derivative H—X—D—X—H is chosen from diols of structure: 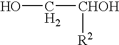
R² being chosen from alkyl chains having from 8 to 40 carbon atoms.

36. A cosmetic composition according to claim 20, wherein said diamine dimers are chosen from branched C₃₆ aliphatic and alicyclic diamines and combinations of derivatives obtained from the conversion of the corresponding dimeric fatty acids.

37. A cosmetic composition according to claim 36, wherein said diamine dimer is obtained from the conversion of dimeric fatty acids.

38. A cosmetic composition according to claim 20, wherein said difunctional derivative H—X—D—X—H is chosen from diamines of structure
H₂N—D—NH₂
in which D is chosen from linear and branched alkyl chains containing from 8 to 40 carbon atoms.

39. A cosmetic composition according to claim 10, wherein said monofunctional derivatives A₁—X—H and A₂—X—H are chosen from
linear and branched monoalcohols and monoamines having alkyl chains containing 1 to 80 carbon atoms,
natural and synthetic oils bearing only one hydroxyl group per chain,
glycerol diesters, and
citric acid triesters of fatty alcohols.

40. A cosmetic composition according to claim 10, wherein said polymerization reaction is carried out in the presence of additional reagents chosen from
(a) polyorganosiloxanes of structure: 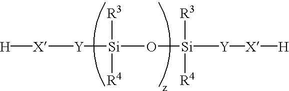
in which
Z denotes an integer ranging from 0 to 100,
X′, which may be identical or different, is chosen from —O—, -and NH—,
Y, which may be identical or different, is chosen from hydrocarbon-based radicals of structure —(CH₂)ₐ—, with “a” ranging from 0 to 10, and
R³ and R⁴, which can be identical or different, are groups chosen from methyl, phenyl, alkyl, perfluoroalkyl, and polyoxyalkylene groups,
(b) polyethers of structure H—X′—E—X′—H, in which
X′, which may be identical or different, is chosen from —O—, and —NH—, and
E is chosen from polyoxyalkylene chains,
(c) polyesters of structure HO—F—OH in which F is a block chosen from aliphatic and aromatic polyester blocks,
(d) polyamides of structure H₂N—G—NH₂ in which G is chosen from polyamide blocks,
(e) polyorganosiloxanes comprising a single difunctional end, of structure 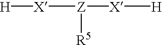
in which
X′, which may be identical or different, is chosen from —O— and —NH—,
Z is chosen from hydrocarbon-based trivalent radicals which may contain at least one hetero atom, and
R⁵ is a polyorganosiloxane segment,
(f) difunctional coupling agents of structure H—X′—X′—H in which X is chosen from —O— and —NH—, and J is chosen from linear, branched, and cyclic hydrocarbon-based divalent radicals.

41. A cosmetic competition according to claim 40, wherein said polyamide block is chosen from polyamides resulting from the reaction of a fatty acid dimer and of a diamine.

42. A cosmetic competition according to claim 40, wherein said polyamide block is chosen from polyamides resulting from the reaction of a fatty acid dimer and of a diamine, said polyamide block being aliphatic.

43. A cosmetic composition according to claim 40, wherein said at least one hetero atom is chosen from oxygen, sulfur, and nitrogen.

44. A cosmetic composition according to claim 40, wherein J is chosen from linear, branched, and cyclic hydrocarbon-based divalent radicals bearing at least one hydrophilic group.

45. A cosmetic composition according to claim 44, wherein said at least one hydrophilic group is chosen from carboxylic acids, sulfonic acids, tertiary amines, and polyoxyalkylene groups.

46. A cosmetic composition according to claim 5, wherein said polymer defined by formula I is chosen from the products of the reaction of two moles of difunctional derivative H—X—D—X—H with one mole of diisocyanate.

47. A cosmetic composition according to claim 5, wherein said polymer defined by formula III is chosen from the products of the equimolar reaction of a difunctional derivative H—X—D—X—H with a diisocyanate, the residual isocyanates being consumed by a monofunctional compound A₁—X—H.

48. A cosmetic composition according to claim 10, wherein said at least one polymer is the product of the reaction of
one mole of compound H—X—D—X—H,
three moles of diisocyanate, and
two moles of coupler of structure 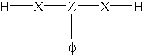
in which φ is chosen from linear, branched, and cyclic aliphatic chains containing from 8 to 20 carbon atoms and said at least one polymer product is a grafted block polymer of formula 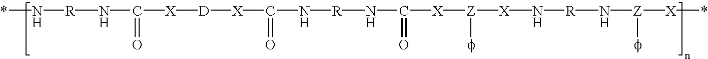
wherein n is an integer ranging from 1 to 1 0,000.

49. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said at least one polymer is present in an amount ranging from 0.1% to 80% of the total weight of the composition.

50. A cosmetic composition according to claim 49, wherein said at least one polymer is present in an amount ranging from 0.5% to 40% of the total weight of the composition.

51. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said at least one hydrocarbon-based oil is chosen from polar oils comprising at least one functional group chosen from esters, ethers, acids, and alcohols.

52. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said at least one hydrocarbon-based oil is chosen from apolar oils.

53. A cosmetic composition according to claim 52, wherein said at least one hydrocarbon-based oil is chosen from at least one of parleam oil, squalane and aliphatic hydrocarbons.

54. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said at least one fatty phase comprises not more than about 40% of silicone oil.

55. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said cosmetic composition is wax-free.

56. A care, treatment, or make-up-removal composition for a keratin material comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

57. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters, and
further comprising at least one cosmetic active agent.

58. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters, and
further comprising at least one additive chosen from water, antioxidants, essential oils, preserving agents, neutralizers, liposoluble polymers, fillers, fragrances, and cosmetic and dermatological active agents.

59. A cosmetic composition according to claim 57, wherein said at least one additive is present in a proportion ranging from 0 to 20% of the total weight of the composition.

60. A cosmetic composition according to claim 59, wherein said at least one additive is present in a proportion ranging from 0 to 10% of the total weight of the composition.

61. An anhydrous cosmetic composition comprising a thickened to solidified solution wherein said anhydrous cosmetic composition further comprises at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

62. An anhydrous cosmetic composition according to claim 61, wherein said anhydrous cosmetic composition is a gelled solution.

63. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters, and
further comprising water, said cosmetic composition being in the form of an emulsion.

64. A cosmetic composition according to claim 63, wherein said cosmetic composition is in the form of a water-in-oil emulsion.

65. A cosmetic composition according to claim 64, wherein said water-in-oil emulsion is thickened to solidified.

66. A cosmetic composition according to claim 64, wherein said water-in-oil emulsion is gelled.

67. A cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
wherein said cosmetic composition is packaged in the form of a stick or a dish.

68. A cosmetic composition according to claim 1, further comprising at least one dyestuff.

69. A cosmetic composition according to claim 68, wherein said at least one dyestuff is at least one chosen from lipophilic dyes, hydrophilic dyes, pigments and nacres.

70. A cosmetic composition according to claim 69, wherein said at least one dyestuff is present in a proportion ranging from 0.01% to 40% relative to the total weight of the composition.

71. A cosmetic composition according to claim 70, wherein said at least one dyestuff is present in a proportion ranging from 5% to 25% relative to the total weight of the composition.

72. A cream mascara, an anhydrous mascara, a foundation, a make-up product for the body, an eyeliner or a lipstick comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

73. A method of caring for, making-up, or treating a keratin material of human beings, said method comprising applying a cosmetic composition to the keratin material, said cosmetic composition comprising at least one continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

74. A method of structuring a fatty phase in a cosmetic composition comprising at least one continuous liquid fatty phase containing at least one hydrocarbon-based oil comprising contacting said at least one continuous liquid fatty phase with at least one polymer chosen from polyurethanes, polyurethaneureas, and polyureas, said at least one polymer being soluble or dispersible in said at least one hydrocarbon-based oil, and wherein said at least one polymer comprises in the chain thereof and/or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters.

75. A method of structuring a fatty phase in a cosmetic composition comprising at least one continuous liquid fatty phase containing at least one hydrocarbon-based oil comprising contacting said at least one continuous liquid fatty phase with at least one polymer chosen from polyurethanes, polyurethaneureas, and polyureas, said at least one polymer being soluble or dispersible in said at least one hydrocarbon-based oil, and wherein said at least one polymer is chosen from polymers of formulae I, II, and III: 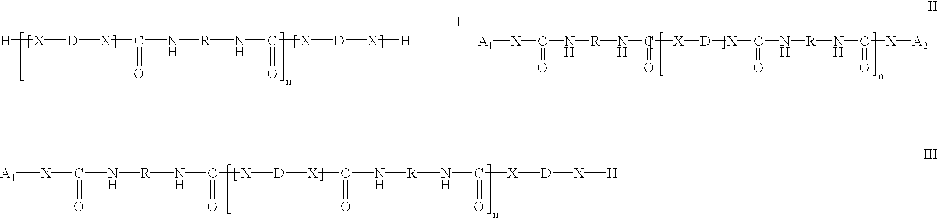
in which:
n denotes an integer ranging from 1 to 10,000,
X, which may be identical or different, is chosen from, —O—, —NH—, and combinations thereof,
R, which may be identical or different, is a divalent radical chosen from alkylene, cycloalkylene and aromatic radicals, and combinations thereof, which are optionally functionalized
A₁ and A₂, which may be identical or different, are chosen from saturated and unsaturated, linear, branched and cyclic monovalent hydrocarbon-based radicals containing from 1 to 80 carbon atoms,
D, which may be identical or different, is chosen from
(1) saturated and unsaturated, aliphatic and cycloaliphatic hydrocarbon-based divalent blocks,
(2) hydrocarbon-based long-chain aliphatic polyester blocks,
(3) grafts 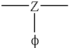
in which Z is chosen from hydrocarbon-based trivalent radicals which may contain at least one hetero atom, and φ is chosen from linear, branched, and cyclic aliphatic chains, and
(4) combinations of the blocks (1) and grafts (3).

76. A method according to claim 75, wherein n denotes an integer ranging from 1 to 1000.

77. A composition comprising a continuous liquid fatty phase containing at least one hydrocarbon-based oil and structured with at least one polymer chosen from polyurethanes, polyurethaneureas, and polyureas, said at least one polymer being soluble or dispersible in said at least one hydrocarbon-based oil, and wherein said at least one polymer comprises in the chain or grafted thereto:
at least two groups chosen from urethane groups and urea groups, and
at least one hydrocarbon-based unit chosen from hydrocarbon blocks and grafts, and from blocks and grafts of hydrocarbon-based, long-chain aliphatic polyesters,
said composition being physiologically acceptable and giving deposits that are at least one of migration-resistant, resistant to water, saliva, sweat, sebum, and/or tears, transfer-resistant, and prolonged staying power after application.
